(19) United States
(12) Patent Application Publication
(10) Pub. No.: US 2011/0087126 A1
(43) Pub. Date: Apr. 14, 2011

(54) Light-Proof Electrodes

(75) Inventors: Anthony Zorzos, Cambridge, MA (US); Clifton Fonstad, Arlington, MA (US); Edward Boyden, Cambridge, MA (US); Giovanni Talei Franzesi, Boston, MA (US); August Dietrich, Somerville, MA (US)

(73) Assignee: MASSACHUSETTS INSTITUTE OF TECHNOLOGY, Cambridge, MA (US)

(21) Appl. No.: 12/824,805

(22) Filed: Jun. 28, 2010

Related U.S. Application Data
(60) Provisional application No. 61/249,733, filed on Oct. 8, 2009.

(51) Int. Cl. A61B 5/04

(52) U.S. Cl. 600/544

(57) ABSTRACT

According to principles of this invention, the photoelectrochemical effect (“PE effect”) may be greatly reduced or eliminated, even when an electrode is immersed in an electrolyte and exposed to light, by using a transparent conductor to record electrical activity. Thus, an electrode with a clear conductor may be used to accurately record electrical activity of neurons and other cells that are exposed to light in vivo or in vitro. Such an electrode eliminates or greatly reduces the artifacts that would otherwise be caused by light due to the PE effect.

### RELATED APPLICATIONS

This application claims the benefit of U.S. Provisional Application Ser. No. 61/249,733, filed Oct. 8, 2009, the entire disclosure of which is herein incorporated by reference.

### STATEMENT REGARDING FEDERALLY SPONSORED RESEARCH OR DEVELOPMENT

This invention was made with U.S. government support under National Institute of Health grants 1RC1 MH088182 and 1R01NS067199, under National Institute of Health Director's New Innovator Award DP2OD002002, and under National Science Foundation grants 0835878 and 0848804. The government has certain rights in this invention.

### FIELD OF THE TECHNOLOGY

The present invention relates generally to electrodes.

### BACKGROUND

A problem with standard electrodes used in neuroscience research is that, when they are immersed in an electrolytic solution and exposed to light, they are subject to artifacts due to the photoelectrochemical effect (the “PE effect”). The PE effect is also known as the Becquerel effect.

Prior to this invention, it was not known how to accurately record electrical activity of neurons or other cells in vivo when the neurons or other cells were exposed to light. More generally, it was not known how to accurately record electrical activity when electrodes are exposed to light and immersed in an electrolytic solution. In both cases, a problem is that the light creates artifacts due to the PE effect.

One reason that this problem is important is that accurate measurement of electrical activity in neurons or other cells in vivo, without corruption due to light exposure, is important for phototherapy.

As background, it is helpful to understand recent advances in phototherapy. Recently, optogenetic reagents have been used to facilitate optical control of neural circuits. These reagents include channelrhodopsin-2 (ChR2), N. pharaonis halorhodopsin (Halo/NpHR), a variant of halorhodopsin, ss-Prl-Halo (sPHalo), an opsin (Arch) derived from an archaebacterium, and an opsin (Mac) derived from the fungus Leptosphaeria maculans. For example, a neuron that has been exposed to such a reagent may, upon exposure to a certain wavelength of light, be activated or silenced.

Using these reagents, it is possible to record spiking activity concurrently with optical neuromodulation without the fast artifact that commonly results from electrical stimulation.

However, it has been widely reported that metal electrodes undergo a slow artifact under exposure to light while immersed in brain tissue (or saline), resulting in electrical signals in the range of Hz to tens of Hz, thus obscuring the recording of local field potentials or electroencephalography signals. This artifact is consistent with the PE effect.

In addition, existing silicon-based microelectrode array implants, exemplified in the “Michigan probe” developed by R. J. Vetter et. al., are fabricated from doped poly-silicon and metal, and, because of the types of materials used, are subject to artifacts from photoelectric interaction.

### SUMMARY

According to principles of this invention, the PE effect may be greatly reduced or eliminated, even when an electrode is immersed in an electrolyte and exposed to light, by using a transparent conductor to record electrical activity. The underlying physics of why this occurs is not fully understood. However, a key inventive insight was that the interaction of light with a conductor would be minimized in a transparent conductor, thereby reducing the PE effect. Prototypes of this invention have demonstrated that the PE effect is in fact eliminated or dramatically reduced.

In some embodiments of this invention, a metal electrode that is coated with a transparent conductor is used to record electrical activity of cells in vivo, thereby greatly reducing or eliminating the PE effect that would otherwise arise when the cells were exposed to light. For example, in some prototypes of this invention, a wire electrode is coated with indium-tin-oxide (ITO). This ITO coating is clear and conductive. The ITO-coated electrode acquires an electrical signal with minimal or no artifact due to light (via the PE effect).

In other embodiments of this invention, an electrode array with a transparent conductor (rather than a metal wire coated with a transparent conductor) is microfabricated. The microfabricated array is used to record electrical activity of cells in vivo, thereby greatly reducing or eliminating the PE effect that would otherwise arise when the array is exposed to light. In the microfabrication process, an arbitrary geometric pattern for the array of electrodes can be imparted onto the conducting material, with resolution limited only by the lithographic limits inherent to microfabrication techniques. In some prototypes of this invention, a microfabricated electrode array uses ITO as a conductor.

Here are some examples of how this invention may be implemented:

This invention may be implemented as a process that comprises using at least one electrode with a substantially transparent conductor to record electrical activity while at least one electrode is exposed to light and immersed in an electrolytic solution. Furthermore: (1) the conductor may comprise ITO, (2) the conductor may comprise at least one of the following: carbon nanotubes, graphene-carbon nanotube hybrid (G-CNT), doped ZnO, SnO₂, and In₂O₃, (3) at least one electrode may comprise a metal wire substrate coated, at least in part, with a substantially transparent conductor, (4) the recording may be performed in vivo, (5) the recording may be of electrical activity of at least one neuron or other biologic cell, (6) for at least one frequency, pulse rate and intensity of said light, the peak-to-peak PE artifact of the coated metal wire may be at least 70% less than said peak-to-PE artifact would be if the metal wire were not coated and were in direct contact with said electrolytic solution, (7) at least one electrode comprises a metal substrate that has been coated with ITO by sputter deposition, (8) a plurality of said electrodes, each with a substantially transparent conductor, comprise a microfabricated array of electrodes, (9) for such an array, a substantially transparent conductor may be deposited, with or without at least one intervening layer of insulation, on at least part of a substrate that comprises silicon, (10) the recording may be of a periodic electrical signal with a frequency of less than 100 Hertz, and (11) the exposure to light may occur during only part of the total duration of said recording.

This invention may be implemented as a method comprising use of an electrode with a substantially clear conductor to record electrical activity of at least one neuron or other cell in such a manner that, during at least part of the duration of said recording, the electrode is exposed to light and immersed in an electrolytic solution. Furthermore: (1) the conductor may comprise ITO, (2) the electrode may comprise a metal wire coated with said substantially clear conductor, and (3) the electrode may be part of a microfabricated electrode device comprising a plurality of electrodes with substantially clear conductors.

This invention may be implemented as an electrode which comprises a metal wire coated with a clear conductor and which is adapted for recording electrical activity while exposed to light and immersed in an electrolytic solution. Furthermore, (1) the clear conductor may comprise ITO that has been coated on said metal wire substrate by sputter deposition, and (2) the clear conductor may comprise carbon nanotubes, graphene-carbon nanotube hybrid (G-CNT), doped ZnO, SnO₂ or In₂O₃.

This invention may be implemented as a microfabricated apparatus comprising a plurality of electrodes and a silicon substrate, wherein at least one electrode comprises a substantially transparent conductor and is adapted for recording electrical activity of a neuron or other cell in vivo.

### BRIEF DESCRIPTION OF THE DRAWINGS

FIG. 1 shows a cross-section of a metal wire coated with ITO, in a prototype of this invention.

FIG. 2 shows a cross-section of a material stack of an electrode array, prior to microfabrication, in a prototype of this invention.

FIG. 3 is a top view of an electrode array, in a prototype of this invention.

FIG. 4 is a top view of a portion of an electrode array, in a prototype of this invention.

### DETAILED DESCRIPTION

According to principles of this invention, the PE effect may be greatly reduced or eliminated, even when an electrode is immersed in an electrolyte and exposed to light, by using a transparent conductor to record electrical activity. The underlying physics of why this occurs is not fully understood. However, a key inventive insight is that if a transparent conductor is used, then interaction between the conductor and light is reduced, which thereby reduces or eliminates the PE effect. Prototypes of this invention have demonstrated that this insight is correct.

For example, this invention may be embodied as (1) a wire electrode coated with a transparent conductor, or (2) a microfabricated electrode array with a transparent conductor. In each case, the coated wire electrode or the electrode array may record electrical activity with minimal or no light artifact (arising from the PE effect).

### Coated Wire Electrodes

First, consider embodiments of this invention in which a wire electrode is coated with a transparent conductor. NiCr wires, PtIr wires and stainless steel wires were used in prototypes of this invention. However, the wire may comprise any other electrode material, such as tungsten or silicon.

In exemplary implementations of this invention, the wire is coated with a clear conductor. In some embodiments of this invention, the transparent conductor is indium-tin-oxide (ITO). ITO has several advantages: (a) ITO can be easily deposited (for example, by using an argon/oxygen rich plasma sputtering technique, as described in more detail below), (b) Significant work has been done to characterize and understand ITO's properties; (c) ITO is biocompatible and works with neural recording, and (d) ITO can be easily etched using common, relatively benign etch chemistries.

However, other transparent conductors may be used instead of ITO. For example, this invention may be implemented with carbon nanotubes, graphene-carbon nanotube hybrid (G-CNT), doped ZnO, SnO₂, or In₂O₃.

A problem that confronted the inventors was how to deposit ITO on the wire substrate. The first method that they tried—dip-coating—turned out to have literally “flaky” results in various prototypes. However, the inventors eventually determined that sputtering is a desirable method of depositing a clear conductor on a wire substrate in some circumstances.

In early prototypes of this invention, dip-coating was used to deposit ITO on a wire electrode as follows: A 50 μm-diameter nichrome wire was dipped in ITO nanoparticles of 20 nm diameter (resuspended to a concentration of 25% in isopropanol) ten times, each time followed by sintering at 500° C. for 30 minutes in air. This dip-coating protocol resulted in wires that responded to light with optical artifacts about 10× lower than normal nichrome, while retaining an impedance similar to electrodes used for neural recordings. Impedances ranged from approximately 0.5MΩ to 7.5MΩ. This dip-coating protocol was chosen in order to enhance mechanical stability of the ITO and adhesion of the ITO to the metal substrate while also reducing the artifact due to PE. However, the results using this dip-coating protocol were highly variable because the dip-coating process is delicate. Prior to sintering, the ITO can fall off the electrode. Also, because the thickness of the ITO layer increases after multiple rounds of sintering, the ITO layer can flake off.

In some later prototypes of this invention, sputtering was used to deposit ITO on electrode wire tips as follows: The wires were taped or otherwise secured to a glass slide. The wire tips were bent at a 90° angle to receive the bulk of the sputtered ITO. A layer of approximately 400 nm of ITO was formed on the tips through sputtering under conditions: platen temperature: 25 C, total plasma pressure: 30 mTorr (Ar partial pressure: 30 mTorr; 100% Ar plasma), 100 W RF power @ 13.56 MHz. The resulting electrodes responded to light with optical artifacts 22× lower than normal nichrome and retained an impedance similar to electrodes used for neural recordings. Impedances ranged from approximately 0.5MΩ to 7.5MΩ. This sputtering protocol produced less variable results and better reduction of PE artifact than the dip-coating protocol.

In many cases, it is desirable to coat the ITO with an insulator, such as polytetrafluoroethylene (sold under the brand name Teflon®) or another polymer. The insulator is not applied at the tips of the wire where electrical recording occurs. Nor is it usually applied on electrical contact pads.

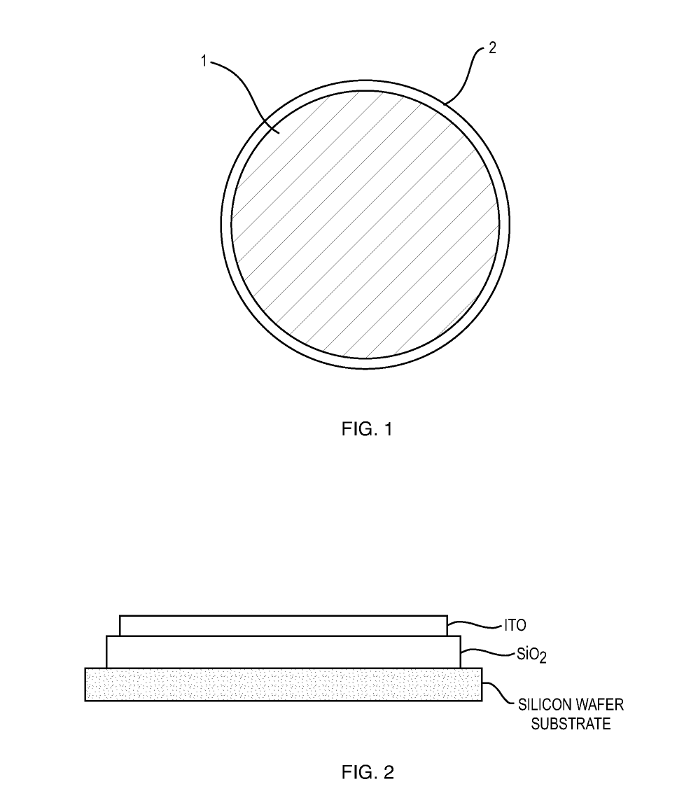

FIG. 1 is a cross-section (not to scale) of an electrode comprising a metal wire substrate (1) coated with ITO (2). The cross-section is of a tip of the electrode where electrical recording occurs, and thus the ITO is not covered with an external layer of insulation.

The design of the wire electrode may be adjusted to meet the needs of the application. For example, the wire diameter may be smaller or larger, the size of the insulating layer may be smaller or larger, the material used for insulation may be any desirable, and the thickness of the layer of ITO deposited onto the electrode tip may be greater or smaller. Other variable properties include electrical insulation properties, adhesion, mechanical stability, and optical properties. The shape of the device need not be long and cylindrical like a standard wire electrode. It may take any shape, depending on the needs of the application of the invention. Also, the device may function regardless of whether or not it is under illumination at the time.

Further, the method of depositing the transparent conductor may be varied, depending on the needs of the application. For example, if the application requires only straight wires, then e-beam evaporation may be used for ITO deposition. However, in the case of e-beam evaporation, any small angle in the wire yields a significantly non-uniform coating. (In contrast, sputtering, as a high-pressure process, yields significantly more uniform coatings).

The efficacy of this invention has been demonstrated on NiCr wires, PtIr wires, and stainless steel wires. For example, such wires, when coated with ITO in accordance with principles of this invention and exposed to blue light flashed at 12.5 Hz, exhibit a marked reduction in PE artifact compared to such wires when they are exposed to such light but not coated.

### Microfabricated Electrode Array

Second, consider embodiments of this invention involving a microfabricated electrode array with a transparent conductor (rather than a metal coated with a clear conductor). Such an electrode array may be used to acquire an electrical signal with little or no interference from light via the PE effect.

In a prototype of this invention, the conductor comprises ITO and the substrate comprises a silicon wafer. Although the ITO layer (and the insulation between the ITO and the substrate) can be deposited onto virtually any general flat surfaced material, silicon is desirable for its balance of availability, compatibility, ease of use, and sturdy mechanical properties. Silicon wafers further offer a simple method by which the device thickness can be controlled.

A layer of insulating material may be deposited between the ITO layer and silicon substrate. The type and thickness of insulating material depends on the device application, where factors of consideration include: potential capacitive coupling, electrical insulation properties, adhesion, mechanical stability, and optical properties. In this prototype, the insulation comprises SiO₂.

In this prototype, both the SiO₂ insulation and ITO are deposited in a plasma-enhanced chemical vapor deposition chamber. This reduces wafer contamination between deposition steps. The electrical properties of the device depend on the quality and thickness of ITO deposited. In this prototype, the ITO layer is 300 nm thick, the SiO₂ insulation is 500 nm, and the silicon substrate is 500 μm thick.

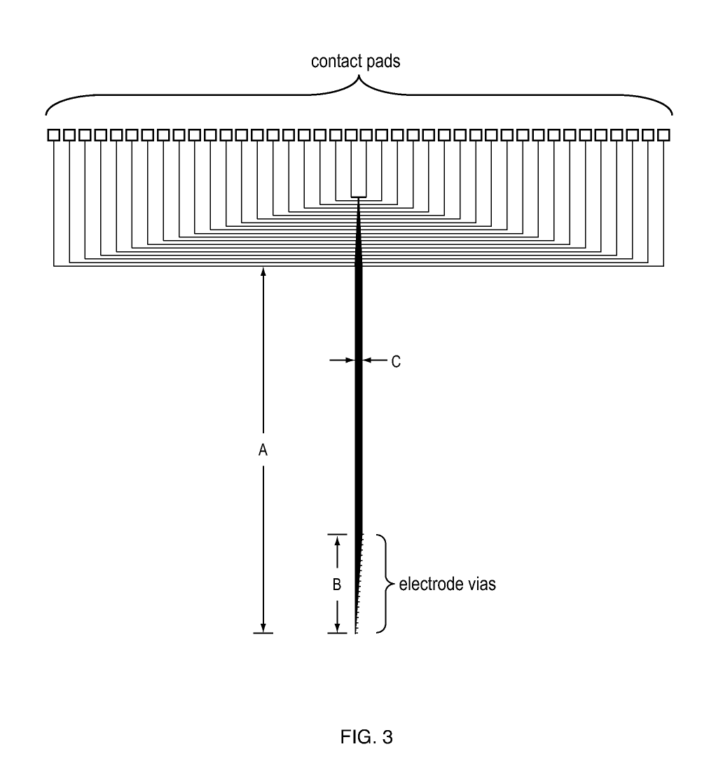

FIG. 2 shows a cross-section of the ITO layer, insulating layer, and silicon substrate layer, prior to microfabrication, in a prototype of a microfabricated electrode array.

A problem that confronted the inventors was how to etch the ITO. In early prototypes, wet etching was used. However, as the size of interconnects become smaller in later prototypes, the undercutting inherent in wet etching became too severe. The inventors found that a dry, more anisotropic etch—such as deep reactive-ion etching (DRIE)—is desirable for applications with small interconnects.

Another problem that confronted inventors was how to remove burnt resist from ITO (after ITO etching). Initially, a piranha etch was tried, but it attacked the metal in the ITO and disadvantageously altered conduction in the ITO. Eventually, the inventors found that a 3-fold wet treatment was optimal for some applications. This wet treatment comprises applying (a) a heated microstrip solution, followed by (b) an O₂ plasma ash, followed by (c) heated microstrip solution.

In some implementations of this invention, the ITO for an electrode array is lithographically patterned in a three-step process: (1) a one micron thin layer of OCG-825 positive photoresist is deposited and patterned, (2) the ITO is etched in a reactive ion etch (RIE) chamber (with the photoresist acting as a mask), where the plasma chemistry is CH₄, H2, and Ar with an RF power supply of 275 watts @ 13.56 MHz and a DC bias of 50 V, and (3) the reticulated resist is removed in a two part piranha etch (1:3, H₂O₂:H₂SO₄).

This invention may be implemented in many different ways as an electrode array. Any photoresist can be used as a mask in the RIE etching process. Furthermore, any material with a necessary selectivity relative to ITO in the RIE chamber (depending, of course, on the ITO thickness) would suffice. It is of note also that the methane-rich plasma described above is not the only chemistry capable of etching ITO; other chemistries may be used if the etching achieves appropriate side-wall etching angle and mask selectivity.

Furthermore, remaining resist may be removed in a variety of ways. For example, in some applications, oxygen-rich plasma “ashing,” organic solvent removal, or mechanical scrubbing. In this specific case, a piranha solution is used for ease and expediency's sake. Furthermore, “dry” plasma etching in general is not necessary for patterning the ITO. Any etching method, including “wet” chemical etching, is possible. The more appropriate etching method is dictated by the geometrical pattern one wishes to transfer to the substrate surface as well as the ITO thickness. A dry etch is desirable for a high aspect ratio design, whereas quicker wet etching techniques will suffice for low aspect ratios.

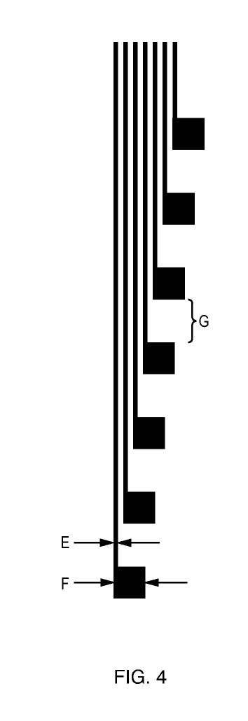

FIG. 3 shows the etched transferred pattern in the ITO, in a prototype of this invention. The overall geometry of ITO is apparent in this pattern. As shown in FIG. 3: The shank length (A) is 6 mm, the shank width (C) is 160 μm. The shank bevels to a 20 μm tip over a range (B) of 2 mm. The 40 electrode sites are 20 μm×20 μm squares, separated by 30 μm vertically and 4 μm horizontally,

FIG. 4 is a diagram of a small portion of the shank shown in FIG. 2. Specifically, it shows the beveled bottom of the shank, including some electrode sites (depicted as squares) that are at or near the bottom.

In the prototype shown in FIGS. 3 and 4, the interconnects connecting the electrode sites to the external contact pads are 2 μm wide (E) separated by 2 μm. Each electrode site is 20 μm wide (F). The electrode sites are separated from each other by 30 μm (G). The 200 μm contact pads are separated by 100 μm and aligned in a row. The geometry of the pattern can be arbitrarily varied. The geometry of the prototype shown in FIGS. 3 and 4 is appropriate for certain applications in neuroscience research. However, depending on the application, other geometries may be used, with resolution limited only by the lithographic limits inherent to microfabrication techniques.

In this prototype, the topside insulating material used for insulating the interconnects and electrode sites from one another is 200 nm of SiO₂. The SiO₂ over the electrodes and contact pad region is then removed using a similar process as the ITO etching: (1) deposit and pattern an etch mask, (2) etch targeted regions, and (3) remove mask material. As with the previous ITO etching, the mask material, etch methodology, and mask removal procedure can all take on various forms, depending on the device application. Furthermore, as with the underlying insulation material, the top-side insulator can also take on many forms depending on the design constraints.

The overall probe structure is then removed from the silicon wafer. In this prototype, this is accomplished with a deep reactive ion etch (DRIE) tool. A backside aluminum hard mask is front-to-back aligned and used in a DRIE etch. This is accomplished by (1) depositing and front-to-back aligning an image-reversal AZ5214 photoresist, (2) depositing a thin 50 nm film of aluminum, (3) lifting off the sacrificial photoresist layer, (4) through-wafer etching the silicon wafer, and (5) removing the aluminum hard mask with a phosphoric-acetic-nitric (PAN) wet etch. Again, this is one way among many these probe structures can be isolated from their substrate. For example, there are many sacrificial layers that can be used for a “lift-off” procedure, there are many hard mask materials and thicknesses that will suffice, and there are many capable through-wafer etching procedures and chemistries. Furthermore, one can engage in isolation techniques separate from plasma etching, including laser cutting, chemical etching, and mechanical sawing.

In a prototype implementation of this invention, the device is then packaged to a connector as follows: The packaging method, whereby the contact pads are electrically connected to arbitrary electrical leads, involves the use of an anisotropic conducting tape. The tape is applied over the device contact pads, the arbitrary connector leads are then aligned to those contact pads and bonded via pressure and heat treatments. Any connection method whereby the contact pads are put into electrical contact with connector leads is viable. These connectors are then fed to devices designed for reading electrical dynamics.

Polyethylene terephthalate (PET) may be used as the substrate, instead of silicon. This stiff polymer is commonly used in conjunction with ITO films in the flexible organic light-emitting-diode (OLED) community. PET films are bio-compatible and stiff enough for implantation at relevant probe spatial scales. A further advantage is that PET is non-conducting and will not need an insulation layer before the ITO layer.

In illustrative embodiments of this invention, an electrode array is microfabricated and has micron-scale features, as described above. However, this invention is not limited to that scale.

The electrode sites in the electrode array can be arbitrarily sized, placed, numbered, and ordered to fit specific application needs.

### Applications

A key advantage of the embodiments of the invention discussed above—including a coated wire electrode and a microfabricated electrode array—is that they can be used to record electrical activity while avoiding corruption from an external light source. As a result, they have many practical applications.

For example, this invention may be used to achieve a great reduction of PE artifact for an electrode that is recording electrical activity in brain tissue.

More generally, this invention may be implemented to record electrical activity with little or no PE artifact in any electrolyte medium, whether in vivo, in vitro, or otherwise.

For example, this invention may be implemented to allow accurate recording of electrical activity of cells (e.g., cardiac cells, muscle cells, cells in culture, cells for drug screening), under light activation. A standard electrode cannot accurately measure Local Field Potentials (LFPs) and other electrical potentials (e.g., muscle potentials) shifting at less than 100 Hz in an artifact-free way, because of the PE effect. But a light-proof electrode can. A light-proof wire electrode, implemented in accordance with the principles of this invention, may be used to advantage in monitoring treatments of disorders (such as Parkinson's Disease) that may be characterized by LFP variation.

More generally, light-proof electrodes may be used for monitoring voltage in any environment with an aqueous medium and light. They can also be used for environmental monitoring, solar energy voltage monitoring, and other fields outside of biomedicine.

A light-proof electrode, implemented in accordance with the principles of this invention, may be used to advantage for, among other things, neural probes, display technologies, touch-pad interfaces and solid-state lighting

In some embodiments, this invention may be used to facilitate phototherapy. Targeted, cell-specific phototherapy offers therapeutic promise. Researchers have recently found that, by using light and optogenetic reagents, excitable cells (heart cells, brain cells, etc.) can be activated or silenced, or have their pH altered, to produce long-term cell activity alteration. A large number of neurological, psychiatric, cardiac, and metabolic disorders (such as epilepsy and Parkinson's disease) can potentially be treated by phototherapy. Light-proof electrodes, implemented in accordance with this invention, may be used to facilitate such phototherapy by accurately recording electrical activity (within little or no PE effect) even when illuminated and immersed in an electrolytic solution.

This invention may be used to advantage for observing electrical potentials shifting at less than 100 Hz.

### CONCLUSION

It is to be understood that the methods and apparatus which have been described above are merely illustrative applications of the principles of the invention. Numerous modifications may be made by those skilled in the art without departing from the scope of the invention. The scope of the invention is not to be limited except by the claims that follow.

What is claimed is:

1. A process that comprises using at least one electrode with a substantially transparent conductor to record electrical activity while at least one said electrode is exposed to light and immersed in an electrolytic solution.

2. The process of claim 1, wherein said conductor comprises ITO.

3. The process of claim 1, wherein said conductor comprises at least one of the following: carbon nanotubes, graphene-carbon nanotube hybrid (G-CNT), doped ZnO, SnO₂, and In₂O₃.

4. The process of claim 1, wherein at least one said electrode comprises a metal wire substrate coated, at least in part, with a substantially transparent conductor.

5. The process of claim 4, wherein said recording is performed in vivo.

6. The process of claim 4, wherein said recording is of electrical activity of at least one neuron or other biologic cell.

7. The process of claim 4 wherein, for at least one frequency, pulse rate and intensity of said light, the peak-to-peak PE artifact of said coated metal wire is at least 70% less than said peak-to-PE artifact would be if said metal wire were not coated and were in direct contact with said electrolytic solution.

8. The process of claim 4, wherein at least one said electrode comprises a metal substrate that has been coated with ITO by sputter deposition.

9. The process of claim 1, wherein a plurality of said electrodes comprise a microfabricated array of electrodes.

10. The process of claim 8, wherein said substantially transparent conductor is deposited, with or without at least one intervening layer of insulation, on at least part of a substrate that comprises silicon.

11. The process of claim 1, wherein said recording is of a periodic electrical signal with a frequency of less than 100 Hertz.

12. The process of claim 1, wherein said exposure to light occurs during only part of the total duration of said recording.

13. A method comprising use of an electrode with a substantially clear conductor to record electrical activity of at least one neuron or other cell in such a manner that, during at least part of the duration of said recording, said electrode is exposed to light and immersed in an electrolytic solution.

14. The method of claim 13, wherein said conductor comprises ITO.

15. The method of claim 13, wherein said electrode comprises a metal wire coated with said substantially clear conductor.

16. The method of claim 13, wherein said electrode is part of a microfabricated electrode device comprising a plurality of electrodes with substantially clear conductors.

17. An electrode which comprises a metal wire coated with a clear conductor and which is adapted for recording electrical activity while exposed to light and immersed in an electrolytic solution.

18. The electrode of claim 17, wherein said clear conductor comprises ITO that has been coated on said metal wire substrate by sputter deposition.

19. The electrode of claim 17, wherein said clear conductor comprises carbon nanotubes, graphene-carbon nanotube hybrid (G-CNT), doped ZnO, SnO₂ or In₂O₃

20. A microfabricated apparatus comprising a plurality of electrodes and a silicon or PET substrate, wherein at least one said electrode comprises a substantially transparent conductor and is adapted for recording electrical activity of a neuron or other cell in vivo.
